(19) United States
(12) Patent Application Publication
(10) Pub. No.: US 2013/0204181 A1
(43) Pub. Date: Aug. 8, 2013

(54) DUAL-MODAL IMAGING-GUIDED DRUG VEHICLE WITH ULTRASOUND-TRIGGERED RELEASE FUNCTION

(71) Applicant: Tse-Ying LIU, Taipei City (TW)

(72) Inventor: Tse-Ying LIU, Taipei City (TW)

(21) Appl. No.: 13/546,474

(22) Filed: Jul. 11, 2012

(30) Foreign Application Priority Data
Feb. 2, 2012 (TW) 101103360

(51) Int. Cl. A61M 1/00; A61K 9/14

(52) U.S. Cl. 604/28; 424/489

(57) ABSTRACT

Present invention relates to a dual-modal imaging (magnetic resonance and ultrasound)-guided drug vehicle and system, which possess encapsulation of hydrophobic drug and ultrasound-triggered release function. In the delivery system of the invention, the drug vehicle carrying certain drug (or drugs) is detectable by magnetic resonance imaging, and the release of drug is triggered by ultrasonication when the drug vehicle arrives at target site and accumulates to a desirable concentration. The MRI-guided drug delivery system provides improved accuracy of drug releasing, including position and timing.

### BACKGROUND OF THE INVENTION

This invention relates to an imaging-guided drug vehicle and system with dual-modal imaging (magnetic resonance imaging and ultrasound imaging) and ultrasound-triggered release functionalities. The characteristic of the invention is using magnetic resonance imaging to track the position of the drug vehicle and trigger the drug release using medical ultrasound in vitro when the drug vehicle arrives at the target site and attains sufficient density.

### FIELD OF PRIOR ART

Current ultrasound image-guided drug delivery system mainly uses commercial ultrasound contrast agent microbubble to carry drugs, for which the ultrasound contrast agent may perform the function of trafficking and ultrasound-triggered drug release. However, the mentioned drug delivery system might encounter following issues: (1) In the process of ultrasound imaging, the ultrasound may “see” the contrast agent at non-affected area in a series of image probing and the drug may also be released by the triggering at non-affected area, which makes the mentioned drug delivery method more difficult to control the timing and location of release; (2) It is difficult for microbubbles to load sufficient drug, especially to load sufficient hydrophobic drugs; (3) the image resolution of ultrasound is not high enough for molecular imaging. Based on the issues stated above, in addition to medical diagnostic ultrasound, it is necessary to find a new medical imaging technology for vehicle's tracking.

The conventional ultrasound image-guided drug delivery system often uses albumin-based (Duvshani-Eshet, M. etc. Journal of Controlled Release 112, 156-166 (2006)) or lipid-based (Vlaskou D etc. Advanced Functional Materials 20, 3881-94 (2010)) microbubbles as ultrasound contrast agent and drug vehicle. However, since the ultrasound imaging has to go through a series of image probing, the microbubble may be triggered and explode before reaching the target site. Or, the drug may be released during ultrasound probing even the density of the vehicle is below the target value. The conventional “ultrasound image-guided drug delivery system” often uses albumin-based or lipid-based microbubbles. While research shows that these bubbles has the magnetic resonance (MR) imaging capacity, the MR imaging effect is quite limited because of the absence of magnetic material and clear MR image can hardly be obtained when the density is low. When albumin-based or lipid-based microbubbles are used in the traditional “ultrasound image-guided drug delivery system”, either the hydrophobic drug is loaded in the interface layer between the microbubble and its interior gas (Tinkov, S. etc., Journal of Controlled Release 148, 368-372 (2010)) or the drug is grafted directly on the shell of the microbubbles (Liu, Y. Y. etc., Journal of Controlled Release 114, 89-99 (2006)). However, both methods can load limited amount of drug and need specific processing technology and equipment. In addition, these two prior arts can not demonstrate MR imaging function.

The production of drug vehicle with MRI function in the conventional technology uses is macromolecular solid sphere or macromolecular micelle to load drug and magnetic nano-particles (Talelli, M. etc., Langmuir 25, 2060-2067 (2009)). However, the imaging vehicle in this method doesn't allow ultrasound triggered drug release.

The contrast agent in the conventional “dual-modal imaging and drug delivery system” has both ultrasound and magnetic resonance imaging functions and can be made of “macromolecular hollow multilayer sphere”. Gas such as SF₆ is filled into the core of the macromolecular hollow sphere to allow ultrasound imagine. Magnetic particles and drugs are embedded in the layers of the macromolecular sphere to allow magnetic resonance imaging and drug delivery (Fang, Y. etc., Biomaterials, 30, 3882-3890 (2009)). However, the shell of this kind of vehicle is too solid to be destructed by ultrasound and thus doesn't perform ultrasound-triggered drug release. Furthermore, the contrast agent of both ultrasound and magnetic resonance imaging functions can be made of microbubbles coated with magnetic nano particles. However, the dual-modal contrast agent provided by this prior art can hardly load hydrophobic drugs (Vlaskou, D. etc., Advanced Functional Materials 20, 3881-3894 (2010); Lee, M. H. etc., Langmuir 26, 2227-2230 (2010)). The microbubble drug-loading method in the present invention is quite different from the 2 prior arts.

The present invention is about an ultrasonically-triggered drug vehicle with magnetic resonance imaging function. While albumin-based or lipid-based microbubbles are used, the characteristic of the present invention is to detect the position and the density of vehicle using magnetic resonance imaging by MR signal produced by magnetic nano particles on the surface of microbubbles. After confirming the right position and timing for drug releasing, the microbubbles are destructed by ultrasound to release drug. Furthermore, in the present invention, the hydrophobic drug is preloaded in the self-assembled nano micelles will later be absorbed on the surface of microbubbles. This is apparently different form the conventional is technology of drug vehicle.

One conventional technology disclosed by the lab of Dr. Chih-Cheng Lu of National Taipei University of Technology uses mainly chemically modified magnetic nano-particles to allow the grafting of the magnetic nano particles on the microbubbles (Taiwan patent no. 096105671, Chih-Cheng LU, Processes for magnetic micro-bubbles). However, this prior art can not encapsulate drug in the microbubbles. Some international studies shows that magnetic nano-particles loaded microbubbles can be formed using flow processing. However, the fragile structure of microbubbles made it hard for magnetic nano-particles to, via in situ synthesis, graft on the surface of the bubbles or in the structure of bubble shells. If chemical process is used to modify magnetic particles and graft using the chemical bonds, three issues can occur: (1) Chemical grafting process might destroy the fragile structure of microbubbles and make them explode or disappear; (2) Magnetic nano-particles will not only attach to the surface of the microbubbles but also enter in the solution surrounding the bubbles. This makes it difficult to distinguish the microbubbles and the unattached magnetic particles and affects the result of MRI; (3) In the prior art, it is difficult to load the magnetic nano particles and drugs on the microbubbles at the same time. The present invention provides a novelty to load both magnetic particles and drugs on the surface of microbubbles without damaging the structure of microbubbles. Its structure and process are both quite different from the prior arts and it doesn't need complex or special equipment.

The microbubbles in the present invention contain SF₆ gas (or equivalent) to allow ultrasound imaging. The shell of the bubbles is made of albumin or lipid. The self-assembled micelles attached on the bubbles contain drugs and magnetic particles to allow magnetic resonance imaging and ultrasound-triggered drug release while using high frequency (1-3 MHz or above) and low power density (0.5 W/cm² or below) of medical diagnostic ultrasound to destruct the drug vehicles.

### SUMMARY OF THE INVENTION

Based on the above-mentioned considerations, this invention provides an ultrasonically triggered drug vehicle with magnetic resonance imaging and ultrasound imaging functions that can load hydrophobic drugs. It tracks the location of drug vehicle using MRI and triggers destruction of vehicle using in vitro therapeutic ultrasound for drug release when the vehicle attains enough density at the target site. The precision of timing and location of drug release can be much improved by this method. The medical effect of chemical treatment can also be increased given the genuine characteristics of ultrasound that are acceleration of penetration of sonophoresis of drugs and increasing tissue temperature.

One characteristic of the present invention is to graft micelles loaded with drugs and superparamagnetic nano-particles on normal ultrasound microbubble contrast agent to encapsulate hydrophobic drugs, perform ultrasonically-triggered release behavior and exhibit MRI function (as shown in FIG. 1). The microbubbles in the present invention contain SF₆ gas (or equivalent) to allow ultrasound imaging. The shell of the bubbles is made of albumin or lipid. The self-assembled micelles attached on the bubbles contain drugs and magnetic particles to allow magnetic resonance imaging and ultrasound-triggered drug release while using high frequency (1-3 MHz or above) and low power density (0.5 W/cm² or below) of medical therapeutic ultrasound to destruct the drug vehicles.

Therefore, this invention provides also a drug vehicle to load hydrophobic drug. It includes microbubbles that can load hydrophobic gas and several micelles attached to the microbubbles. The micelles contains amphiphilic macromolecules, hydrophobic drugs and superparamagnetic particles.

In one of the embodiments of the present invention, the microbubbles can be made of albumin, lipid or other foaming surfactant or macromolecular materials and contain SF₆ or other equivalent gas such as C₃F₈. Depending on the need, the size of the microbubble can be set from 0.3 μm to 10 μm. The microbubbles are with ultrasound imaging function and can be destructed by ultrasound with adequate MI (mechanical index).

In one of the embodiments of the present invention, the microbubbles in the vehicle is filled with SF₆ gas and, when they are triggered by high frequency (1-10 MHz) of ultrasound, the nano micelles on the surface of microbubbles will spread and enter the target site to be absorbed by cells. Also, the vibration wave created after the microbubbles being destructed by high frequency of ultrasound can not only destruct the nano micelles to release the loaded drug, but also create some temporary opening on the cell membrane of the target cells to allow the released drug to enter the cells.

The above-mentioned micelle contains amphiphilic macromolecules, drugs and magnetic nano particles and its particle diameter can be adjusted to 40 nm to 300 nm by processing technology according to the need. The said micelles can be attached on the surface of microbubbles via electrostatic attraction or chemical bonding. Amphiphilic macromolecule has a hydrophilic moiety and a hydrophobic moiety. The hydrophilic moiety allows an even distribution of micelles in the water and the hydrophobic moiety allows the oil soluble superparamagnetic nano particles and hydrophobic drugs to be loaded in the above-mentioned micelles by self-assembly.

In one of the embodiments of the present invention, the above-mentioned self-assembled nano micelles constitute of amphiphilic chitosan and magnetic nano particles. In the self-assembled nano sphere, the micelle structure (hydrophilic outside and hydrophobic inside) allows loading hydrophobic drugs and the magnetic particles allow magnetic resonance imaging. The vehicle has a hydrophilic surface to assure the overall dispersity in the water solution. Furthermore, the charged surface of the micelle makes it easy to graft on the surface of microbubbles via electrostatic attraction. Or the micelle is with surface of NH₂ and COOH to allow graft on the microbubble surface via chemical bonding. Thus the self-assembled nano micelles made of nano magnetic particles can be ultrasonically triggered to release drug and confer magnetic property to microbubbles after grafting on them for subsequent isolating process.

The structure of the drug vehicle in the present invention can be triggered to destruction by high frequency (1-10 MHz) and low density (below 0.5 W/cm²) of ultrasound. The drug-loaded micelles will spread and will also be destructed by both the low frequency (20-500 KHz) of ultrasound and the shock wave of the bubble destruction and then release the hydrophobic drugs (as shown in FIG. 1).

During the absence of ultrasound, the nano particles contained in the micelles of the vehicle of the present invention can stabilize the structure of micelles and avoid the leaking of drugs. After triggered by ultrasound, the micelles can be destructed rapidly because of the weak binding of the self-assembled particles, avoid the re-assembling of macromolecules and release drug rapidly. Besides, before the vehicle is triggered, the above-mentioned magnetic nano particles can provide MRI T₂ imaging contrast. After triggered by ultrasound, the superparamagnetic nano particle distribution will change because of the destruction of microbubbles or micelles. Thus, the different contrast signal of T₂ and T₂* can be observed as a distinction of whether the vehicle is triggered.

### DETAILED DESCRIPTION OF THE INVENTION

The characteristics and advantages of the present invention will furthermore be illustrated and explained in the following preferred embodiments. The preferred embodiments are for better illustration and not for limiting the scope of the present invention.

The description and figures below are to disclose the preferred embodiments according to the present invention. Various modifications may be made to this invention for different usages and situations without departing from the scope covered by the appended claims. People familiar with the common senses in the concerned field can make modification of forms, structures or materials based on the present invention.

### Preferred Embodiments

In one of the preferred embodiments, the micelles on the surface of the drug vehicle contain superparamagnetic Fe₃O₄ (SPIO) nano particles to provide MRI T₂ weighted image (as shown in FIG. 7). The structure of the drug vehicle is ultrasonically triggered to destruction and the distance between the superparamagnetic nano particles and their dispersity will change. The obvious difference of MR signal on the slope difference between R₂* and R₂ can be observed in FIG. 8 and can be the basis for assessing the situation of the vehicle.

As shown in FIG. 2, the structure of the drug vehicle of the present invention contains microbubbles (albumin based or lipid based) that can load hydrophobic gas (such as SF₆ or another equivalent gas such as C₃F₈) in order to attach several micelles (contain amphiphilic macromolecules, drugs and magnetic nano particles) on the microbubbles. The preparation method is described in the following.

First, bovine serum albumin (BSA) microbubbles are prepared. Put 5 mL of glucose solution in a 20 mL sample glass bottle, which is heated in an oil bath pan. Add the BSA powder in the bottle and stir for complete solution and then add 3 mL of glycine. Insert a gas needle in the is bottle to inject SF₆ gas for one minute. Insert an ultrasound homogenizer probe for 3 minutes of ultrasound succussion for the solution to turn immediately from transparent yellow to milky white. Turn off the ultrasound after 3 minutes of succussion, take the sample bottle out of the oil bath and pour the BSA microbubbles in the glutaraldehyde solution and stir for 2 hours. Centrifuge the product at 1000 rpm for 10 minutes to have the bubbles on the upper layer. Retrieve the lower solution with needles to obtain cross-link BSA microbubbles. FIG. 3 shows the conjugate focus microscope image (FIG. 3a) and visible light microscope image (FIG. 3b). It is clearly shown in FIG. 3 that the forms of the microbubbles are complete and only the outer shell is fluorescent green and the darkness inside shows the absence of fluorescent dye that prove the existence of protein only on the shell layer of the microbubbles.

Preparation of N, O-carboxymethyl chitosan (NOCC). Add 50 mL of isopropanol in a 250 mL sing-neck round bottom flask containing 5 g of chitosan. When the chitosan is completely dissolved in the isopropanol, add 12.5 mL 10 N NaOH water solution in 5 times. Within 5 minutes after adding the last time of NaOH, add rapidly 25 g of chloroethanoic acid and stir for 30 minutes. Then, replace the reaction in a 60° C. oil bath pan and stir for 4 hours. After the reaction is completed, place the reaction flask for cooling under room temperature and collect the product by suction filter. Wash the product with methanol and place it in a 65° C. drying oven for 24 hours to obtain water-soluble pale-white N, O-carboxymethylchitosan (NOCC) powder. Place 2 g of the NOCC in a 15 mL reaction flask, add 50 mL of deionized water, stir for a day for complete solution and add 50 mL of methanol and stir for a day for an even mix. Afterward, add 1.4 mL of hexanoyl anhydride for 8 hours of reaction. After the reaction, the yellow transparent and stiff product of carboxymethyl hexanoyl chitosan (CHC) is obtained and dried in a drying oven at 65° C. for 24 hours.

The lipophilic SPIO nanoparticles were mixed with hexane (1 mL) and CHC aqueous solution (0.125% w/v). The mixture was placed in an ice bath and sonicated by probe sonication for 2 min, producing CHC/SPIO micelles. Distract the unloaded drug using column chromatography. Add the nano micelle suspension in the prepared cross-link BSA microbubble solution (containing crosslink agent) and make it electrostatically and chemically (R—CH═N—R bonding) attached on the surface of BSA microbubbles (as shown in FIG. 2). Subsequently, the CHC/SPIO micelle-decorated microbubbles are obtained. The BSA microbubbles decorated with differing amount of CHC/SPIO micelles are named as CHC/SPIO-MB-1X, CHC/SPIO-MB-2X and CHC/SPIO-MB-4X.

The above-mentioned CHC is amphiphilic macromolecule derived from chitosan. It can be dissolved evenly in the water solution because of its hydrophilic carboxymethyl group. And, because of its hydrophobic hexanoyl group, it can load the lipophilic Fe₃O₄ nano particles and hydrophobic drug in the CHC micelle using ultrasound self-assembling.

As shown in FIG. 4, the result from transmission electron microscopy (TEM), the formed micelles contain amphiphilic macromolecule, drugs and magnetic nano particles and the oil phase Fe₃O₄ nano particles are surrounded by CHC and form micelles of 100-200 nm (as shown in FIG. 4a). FIG. 4b is the lattice fringe observed using HR-TEM to confirm those nano particles are Fe₃O₄.

FIG. 5 shows the drug release of the CHC/SPIO micelle-decorated microbubbles after triggered by ultrasound in the preferred embodiment of the present invention. Stimulate the CHC/SPIO micelle-decorated microbubbles by physical therapeutic ultrasound (1 MHz, 0.4 W/cm²) for 20 minutes and distract the drug vehicle using a centrifuge. Add equivalent hexane to destruct the vehicle and extract the drug residue retained in the vehicle. Calculate the drug release rate of the vehicle by measuring the content of drug residue in hexane layer.

As the result shows, 80% of drug release can be attained after 10 minutes of ultrasound stimulus, and 90% of drug release can be attained after 20 minutes of ultrasound stimulus. It means the microbubbles can be destructed by high frequency of ultrasound (frequency 1 MHz, power 0.4 W/cm²) and release the drug contained in the micelles.

In general, the magnetic resonance imaging. T₂ contrast agent is superparamagnetic material such as Fe₃O₄ nano particles which increase T₂ relaxation velocity, extend the T₂ relaxation time and make the T₂ weighted image become darker as the density increases. FIG. 6 is the hysteresis curve of the CHC/SPIO micelle-decorated microbubbles used and prepared in this preferred embodiment. As shown in the figure, CHC/SPIO micelle-decorated microbubbles in this embodiment show hysteresis loop, thus they are both of superparamagnetic and are suitable for magnetic resonance imaging T₂ contrast agent.

As to using superparamagnetic material as T₂ contrast agent, the value of r₂ (the slope of the diagram of density of contrast agent to R₂) is an important index for T₂ contrast agent. The greater r₂ is, the more relevance is between contrast and density. As shown in FIG. 7, the superparamagnetic nano particles contained in the drug vehicle of this invention can provide MRI T₂ contrast imaging before the drug vehicle is triggered by the ultrasound. The vehicle can be tracked using MRI tracking for better control of the drug release location and timing which can be controlled by adjusting amount of SPIO nanoparticles.

Besides, as shown in FIG. 8, the dispersity and the distance between the superparamagnetic Fe₃O₄ nano particles contained in the drug vehicle of the present invention will change significantly (nano particles change from aggregative state to disperse state) after the ultrasound. The magnetic field inhomogeneity established by nanoparticles will also change unevenly and the obvious slope difference of MR signals on the slope between R₂* and R₂ can be observed. The MR signals on difference of slope difference between R₂* and R₂ (r₂*−r₂) will also change greatly, i.e., the slope difference between R₂* and R₂ will lower after the ultrasound stimulus. On the other hand, a comparison of MR images of mouse liver acquired before and after delivery are shown in FIG. 9. As shown, the T₂ contrast of the MR image was enhanced in the liver in the first 20 min after injection. This suggests that the CHC/SPIO-decorated MBs can be employed as a candidate material for MR-image-guided applications. FIG. 10 is the in vivo ultrasound imaging test of injecting CHC/SPIO micelle-decorated microbubbles into SD rat via intravenous injection. As shown in 10a, before the injection of CHC/SPIO micelle-decorated microbubbles, black image was shown in veins and arteries. Right after injection of CHC/SPIO micelle-decorated microbubbles in the iliac vein of rat, a rapid white foggy fluid can be observed in the vein. Several seconds after, the white foggy fluid can be observed in the artery flowing in a pulsing way. The white foggy fluid can also be observed in the liver and the kidney vein of the rat. Based on the results observed, the multi-imaging and ultrasonically triggered drug release constitutes of bubbles and remain the ultrasound-imaging characteristic in vivo and can enter into the body circulation.

To sum up, the drug vehicle in the present invention allows detecting the location and density of the vehicle using MRI and drug releasing using commercial medical therapeutic ultrasound to stimulate the vehicle in order to improve timing of drug delivery at the target site and reduce the intoxication of the chemical treatment to other normal part. Also, the drug vehicle in the present invention is triggered by ultrasound such as medical therapeutic ultrasound to destruct the vehicle to release drug. Currently, the ultrasound is highly commercialized, and the highly safe medical ultrasound has also advantages such as energy focus, precise position orientation and deep penetration of soft tissues. Furthermore, the ultrasound also accelerates drug penetration and absorption and is widely used in clinical therapies such as transdermal drug delivery, hyperthermia and physic treatment. Additionally, in the medical field, clinical experiences of combining diagnostic ultrasound with MRI equipment already exist. The technic in the present invention can be realized using the already existing equipment. This invention is thus practical and safe.

### Other Embodiments

All of the features disclosed in this specification may be combined in any combination. An alternative feature serving the same, equivalent, or similar purpose may replace each feature disclosed in this specification. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### BRIEF DESCRIPTION OF DRAWINGS

FIG. 1 is the structure and function principles of the present invention of “ultrasonically-triggered drug vehicle with MRI function”

FIG. 2 is the preferred embodiment of the present invention. The TEM image of the CHC/SPIO micelle-decorated microbubble (CHC/SPIO-MB) (an albumin microbubble and several nano micelles attached to the microbubble).

FIG. 3 is one preferred embodiment of the present invention, wherein (a) is laser confocal microscope image, and (b) is visible light microscope image of the CHC/SPIO micelle-decorated microbubbles (CHC/SPIO-MB) using BSA microbubbles.

FIG. 4 is the CHC/SPIO micelle-decorated microbubbles of the preferred embodiment of the present invention, wherein (a) is the TEM image of the nano micelles containing amphiphilic macromolecule material (CHC) and superparamagnetic iron oxide nano particles (Fe₃O₄, SPIO) and (b) is the HR-TEM image of superparamagnetic nano particles (Fe₃O₄, SPIO) in nano micelles.

FIG. 5 is the drug releasing of CHC/SPIO micelle-decorated microbubbles of the preferred embodiment of the present invention after triggered by ultrasound.

FIG. 6 is the hysteresis curve of the CHC/SPIO micelle-decorated microbubbles of the preferred embodiment of the present invention. There is no hysteresis loop in either, which shows they are both superparamagnetic.

FIG. 7 is the (a) MRI T₂ image and (b) R₂ mapping of CHC/SPIO micelle-decorated microbubbles (CHC/SPIO-MB-2X, CHC/SPIO-MB-4X) and BSA microbubbles (PMB) of the preferred embodiment of the present invention. We can observe: CHC/SPIO micelle-decorated microbubbles has a high T₂ contrast while BSA microbubbles don't and the relevance between the R₂ value of CHC/SPIO micelle-decorated microbubbles and the density is higher than that of BSA microbubbles.

FIG. 8 Is the R₂−R₂* mapping before and after CHC/SPIO micelle-decorated microbubbles (CHC/SPIO-MB-1X) in the preferred embodiment of the present invention being triggered by ultrasound. We can observe that before (a) and after (b) ultrasound stimulation, the slope difference (r₂*−r₂) of R₂ and R₂* change significantly.

FIG. 9 is the MR image of CHC/SPIO micelle-decorated microbubbles in the preferred embodiment of the present invention before (a) and after (b) being injected in the vein of Balb/c mouse.

FIG. 10 is the intravenous ultrasound image of CHC/SPIO micelle-decorated microbubbles in the preferred embodiment of the present invention before (a) and after (b) being injected in the vein of SD rat.

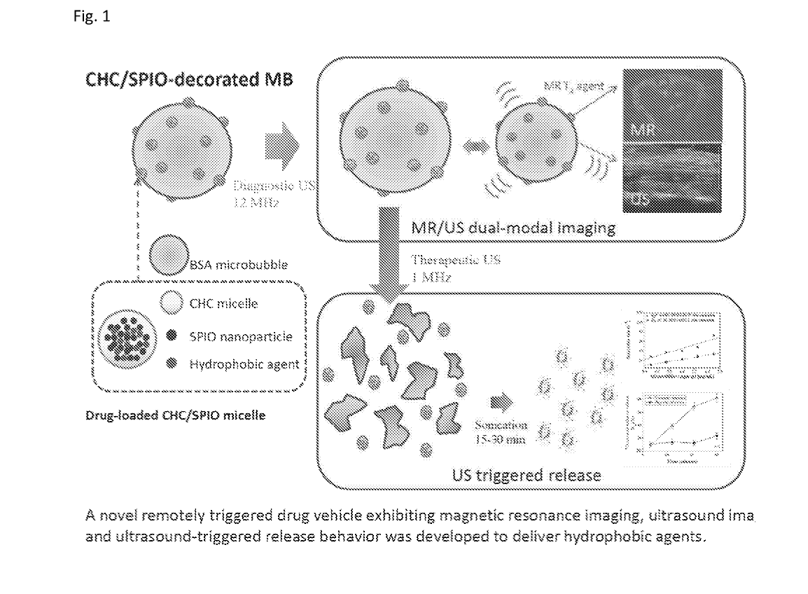

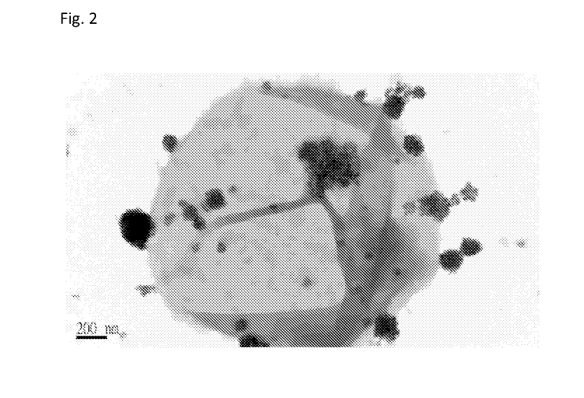

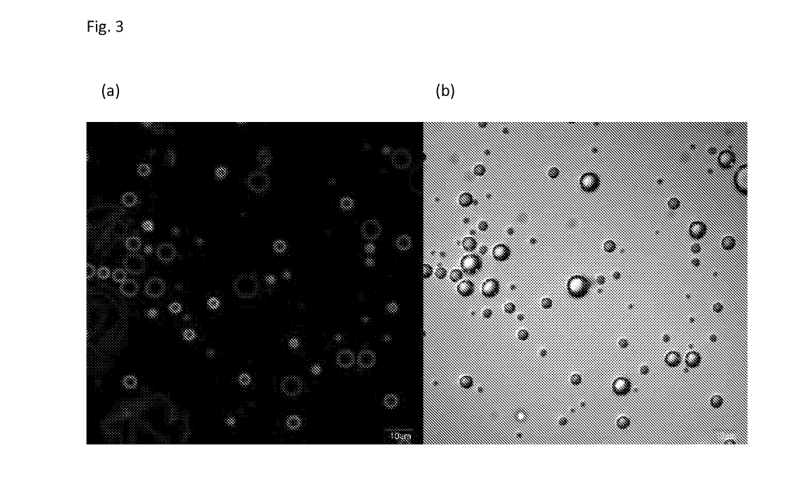

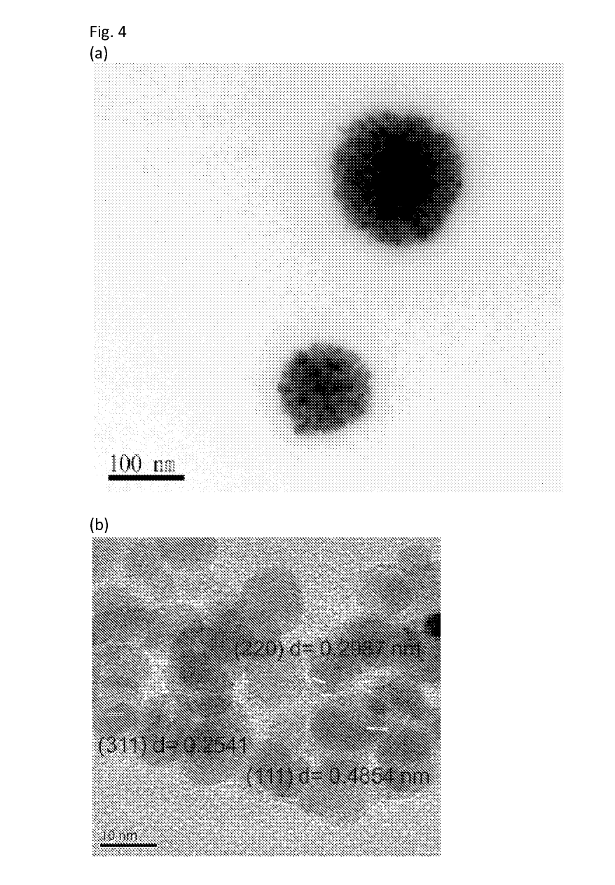

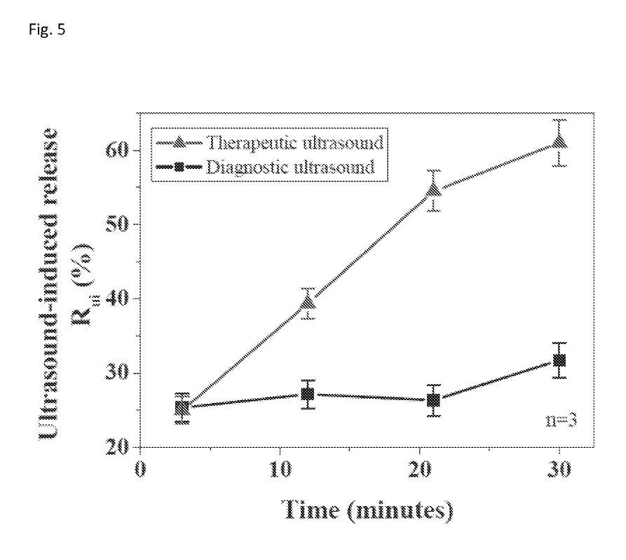

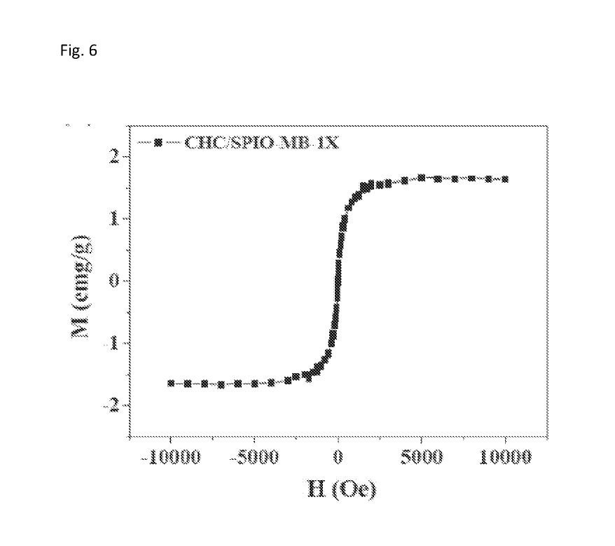

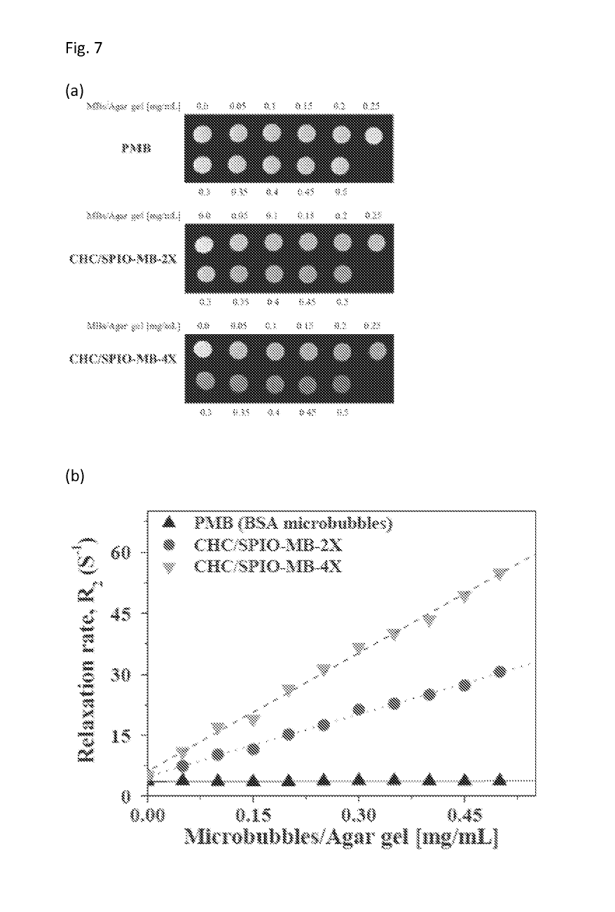

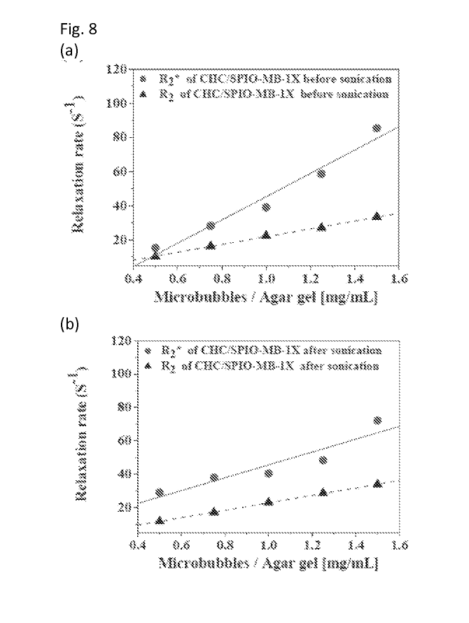

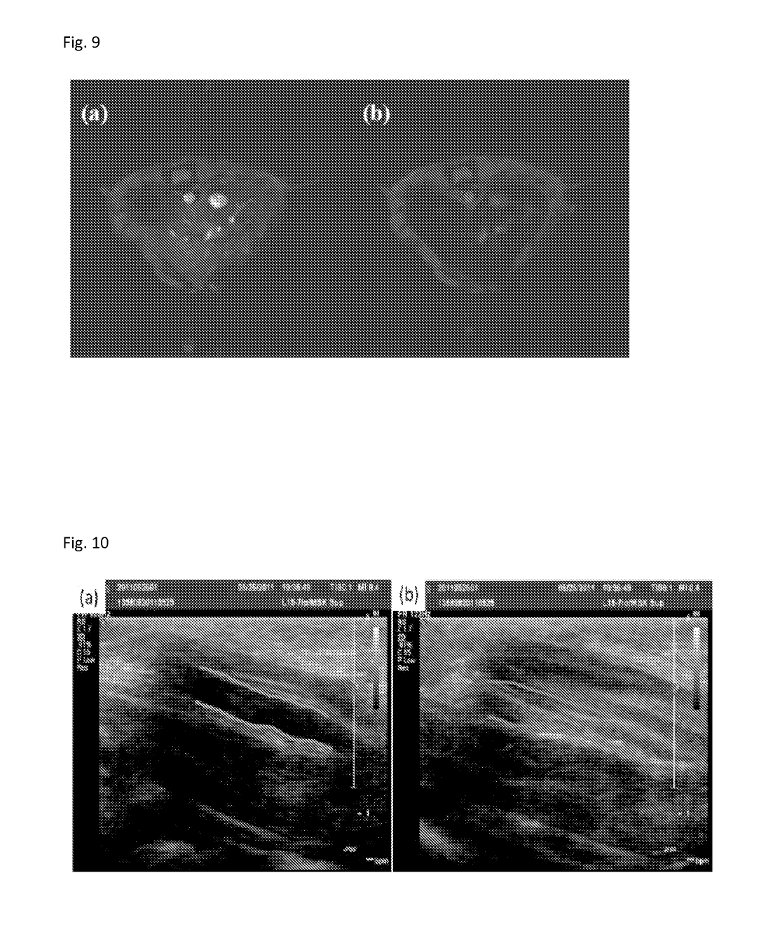

1. An ultrasonically triggered drug vehicle with dual-modal imaging function that loads hydrophobic drugs, which comprises:
A microbubble filled with hydrophobic gas; and
Several nano micelles attached to the surface of the microbubbles. Micelles contain amphiphilic macromolecules, lipophilic superparamagnetic nano particles and hydrophobic drugs.

2. The drug vehicle as in claim 1, wherein the microbubbles are made of albumin, lipid or other foaming surfactant and macromolecular materials and the particle diameter is between 0.3 μm and 10 μm.

3. The drug vehicle as in claim 2, wherein the microbubbles are made of albumin.

4. The drug vehicle as in claim 1, wherein the nano micelles are self-assembled nano spheres made of amphiphilic macromolecular material and superparamagnetic nano particles, the hydrophobic drug is loaded in the hydrophobic inner layer and the particle diameter of micelle are between 40 nm to 300 nm.

5. The drug vehicle as in claim 4, wherein the amphiphilic macromolecular material is carboxymethyl hexanoyl chitosan with a hydrophilic carboxymethyl group and a hydrophobic hexanoyl group.

6. The drug vehicle as in claim 1, wherein the superparamagnetic nano particles are superparamagnetic iron oxide (SPIO) nano particles.

7. The drug vehicle as in claim 1, wherein the drug vehicle is with MRI contrast function that imaging tracking also shows the distribution of vehicles using MRI.

8. The drug vehicle as in claim 1, wherein the characteristics of the nano micelles are that the destruction can be triggered by high frequency and low power density of ultrasound.

9. A drug release method using ultrasonically-triggered drug vehicle, which comprises the following steps:
(1) A drug delivery subject for the drug vehicle as in claim 1;
(2) Image tracking the vehicle using magnetic resonance imaging (MRI); and
(3) When the vehicle attain the target site and cumulate to effective therapeutic density, the ultrasound is used as triggering energy to trigger drug release.

10. The method as in claim 9, where in the vehicle is bombarded by ultrasound of high frequency and low power density which results in cracking the drug vehicle structure without possibility of re-assembling (or re-coating) to release the drug.

11. The method as in claim 9, wherein it further comprises the step of detecting drug release state of the vehicle through the change in magnetic resonance (MR) signal caused by the structural change of the drug vehicle.

12. The method as in claim 11, wherein the change in MR signal refers to the significant change in the difference value between R₂* slope and R₂ slope.

13. A method for preparing the drug vehicle as in claim 1, wherein the nano micelles loaded with drugs and superparamagnetic particles are attached to the surface of an microbubble-based ultrasound contrast agent via electrostatic attraction or chemical bond to form an ultrasonically-triggered drug vehicle with MRI function that can load hydrophobic drugs.
